(19) United States
(12) Patent Application Publication
(10) Pub. No.: US 2019/0209795 A1
(43) Pub. Date: Jul. 11, 2019

(54) VENTILATION APPARATUS FOR CARDIOPULMONARY RESUSCITATION WITH MONITORING AND DISPLAY OF THE MAXIMUM CO2 VALUE MEASURED

(71) Applicant: Air Liquide Medical Systems, Antony (FR)

(72) Inventors: Marceau RIGOLLOT, Montrouge (FR); Jean-Christophe Richard, Antony (FR); Bilal Badat, Paris (FR)

(21) Appl. No.: 16/177,618

(22) Filed: Nov. 1, 2018

(30) Foreign Application Priority Data
Jan. 11, 2018 (FR) 1850224

(51) Int. Cl. A61M 16/00; A61H 31/00; A61M 16/04

(52) U.S. Cl. CPC A61M 16/024; A61M 16/0066; A61H 31/005; A61M 16/0402; A61M 2016/103; A61M 2205/502; A61M 2016/0033; A61M 2202/0208; A61M 2016/0027

(57) ABSTRACT

The invention relates to a medical respiratory assistance apparatus for delivering a respiratory gas such as air, which may or may not be enriched with oxygen, to a patient during cardiopulmonary resuscitation (CPR), having a source (1) of respiratory gas, for example a micro-blower, for delivering a respiratory gas to said patient during cardiopulmonary resuscitation (CPR), and means (4) for measuring the CO₂ content, signal-processing and control means (5), and at least one graphical user interface (7). According to the invention, the signal-processing and control means (5) process the CO₂ content measurement signals, to select the maximum CO₂ content value (Vmax) during a given time period (dt), and to transmit this maximum value (Vmax) to the graphical user interface (7), which displays this maximum CO₂ content value (Vmax).

### CROSS REFERENCE TO RELATED APPLICATIONS

This application claims the benefit of priority under 35 U.S.C. § 119 (a) and (b) to French Patent Application No. 1850224, filed Jan. 11, 2018, the entire contents of which are incorporated herein by reference.

### BACKGROUND

The invention relates to a respiratory assistance apparatus, that is to say a medical ventilator, for delivering a respiratory gas to a patient receiving cardiopulmonary resuscitation (CPR), that is to say a patient in cardiac arrest on whom cardiac massage is performed with alternating compression and relaxation of the chest, with display of the maximum CO₂ content value measured during a given time period.

Medical apparatuses for mechanical ventilation, also called respiratory assistance apparatuses or medical ventilators, are currently used to deliver respiratory gas, for example oxygen-enriched air or non-oxygen-enriched air, to certain patients suffering from respiratory problems.

The delivery of the respiratory gas to the patient is currently effected by means of a motorized and controlled micro-blower, as is described in particular by EP-A-3093498, EP-A-2947328, EP-A-2986856, EP-A-2954213 or EP-A-2102504.

It is known to monitor the gaseous compounds present in the gas administered to the patients, particularly in the gases exhaled by the patients, which gases contain CO₂ resulting from the pulmonary gas exchanges, that is to say CO₂ produced by the patient's metabolism, conveyed to the lungs by the blood stream, then discharged during exhalation by the patient. Thus, etCO₂, standing for End Tidal CO₂ or CO₂ at the end of exhalation, corresponds to the measurement of the CO₂ fraction exhaled in the gases collected during the exhalation of an individual, whether the inhalation is natural or assisted, that is to say obtained by mechanical ventilation.

During mechanical ventilation, different techniques permit spectrophotometric analysis of the CO₂ fraction of the exhaled gases. To do this, the gas present in the exhalation circuit may be:
either aspirated and then analyzed by an analysis cell at a site remote from the respiratory circuit (this procedure is referred to as “sidestream” monitoring),
or analysed near the patient, preferably at a Y-shaped piece arranged in the respiratory circuit in proximity to the patient (this procedure is referred to as “mainstream” monitoring).

During cardiopulmonary resuscitation (CPR) performed on a person in cardiopulmonary arrest, with use of cardiac massage, the alveolar CO₂, which depends not only on the ratios between ventilation and pulmonary perfusion but also on the quantity of CO₂ generated by the cell metabolism, is a very useful parameter for allowing the first responder, for example a physician, to judge the efficacy of the CPR.

In theory, the more effective the CPR, the greater the cardiac output generated by the chest compressions, and the larger the quantity of CO₂ returned to the lungs.

Monitoring of the etCO₂, which indirectly reflects the alveolar CO₂, is increasingly used to monitor the CPR non-invasively, that is to say to provide information to the first responder while performing the cardiac massage, i.e. alternating chest compressions (CC) and relaxations.

FIG. 1 is a capnogram, which is a graphical representation of the variations of the CO₂ content in the respiratory gases of a patient over the course of time (in seconds). This type of capnogram is seen in patients who are ventilated in situations where there is no cardiac arrest. As will be seen, it is divided into four successive phases:
Phase I: This shows the inspiratory baseline, which must be stable at zero.
Phase II: This is the ascending part of the capnogram and corresponds to the appearance of CO₂ in the gases that are exhaled, at the start of the exhalation of the patient, by emptying of the best-ventilated alveoli. In reality, the exhalation begins slightly before this phase, since the gas exhaled at the start of exhalation is devoid of CO₂ because it has not participated in the gaseous exchanges, on account of the instrumental and anatomical dead spaces. The increase in CO₂ is all the slower as the lung is non-homogeneous and the alveoli have long time constants.
Phase III: This corresponds to the alveolar plateau phase which corresponds to the gas rich in CO₂ originating from the least well-ventilated alveoli. The maximum value at the plateau end (PetCO2) corresponds to the etCO₂ value.
Phase IV: This corresponds to the decrease in the CO₂ concentration, caused by the onset of spontaneous or assisted (i.e. mechanical) ventilation.

However, during cardiopulmonary resuscitation (CPR) on a patient in cardiorespiratory arrest, the capnogram is very different for several reasons, notably:
The chest compressions generate movements of small volumes of gas. These volumes, however near the instrumental and anatomical dead space, disturb the capnogram between two ventilatory cycles. Oscillating lines are therefore often observed, since the maximum CO₂ value on each chest compression (CC) does not cease to vary.
The ventilation/perfusion ratios, which are a reflection of the respiratory physiology, are very considerably modified. Moreover, the small gas segments mobilized by the chest compressions may pass the sensor several times. The maximum concentration observed during each chest compression (CC) is thus often far removed from the real alveolar concentration.
Dynamic behaviour of opening and closing of the small airways during CPR has been reported. This phenomenon compromises the exchanges of gas and therefore the interpretation of the CO₂ concentrations during CPR.

It will thus be appreciated that etCO₂ as measured currently, that is to say during each chest compression (CC), does not permit a reliable approximation of the alveolar CO₂, although this alveolar CO₂ is important because it may reflect the quality of the CPR and a possible resumption of spontaneous cardiac activity (RSCA).

The recurring problem that results from this is that a measurement of the CO₂ that does not take account of all or some of these factors, in particular the impact of the ventilation performed on the patient in cardiac arrest and the variability of the CO₂ signal between two machine cycles, makes the use of this CO₂ measurement somewhat unreliable or even unusable.

The current solutions involving the monitoring of etCO₂ are adapted to the CO₂ variations produced by breathing, whether mechanical or spontaneous. The frequencies involved are of the order of 10 to 40 c/min. The algorithms and mechanisms used are adapted to these frequencies and to small variations of the CO₂ between two respirations of the patient.

In this regard, mention made be made of the documents WO-A-2014/072981, US-A-2016/133160 and US-A-2012/016279, which propose methods for monitoring the CO₂ content in the gases exhaled by a patient receiving CPR, in which methods the ventilators indicate that the first responder must stop the cardiac massage when the etCO₂ content is greater than 30 mmHg, for example.

Now, during cardiopulmonary resuscitation, the frequencies of the chest compressions (CC) are close to 100 c/min, the volumes of gas that are mobilized are small, and the gas flow rates are considerable and irregular. Moreover, the problem of the dead space mentioned above adds to these difficulties since, on account of the chest compressions, a same fraction of gas may be analysed several times by the CO₂ sensor, if there is no rinsing or purging of the dead space.

Under these conditions, the etCO₂ value displayed by the current ventilators is refreshed at an inadequate frequency, since the ventilators attempt to follow the evolution of the CO₂ at the massage frequency, i.e. 100 c/min. In other words, the etCO₂ values displayed by the current ventilators are not representative of a CO₂ concentration linked to the patient's metabolism, since the origin of the gas analysed is not guaranteed. In other words, the values measured are often erroneous since they do not reflect, or they reflect very poorly, the concentration of alveolar CO₂.

The problem addressed is therefore to make available an improved respiratory assistance apparatus, that is to say a medical ventilator, with which it is possible, during CPR using the respiratory assistance apparatus, to display a reliable CO₂ value, that is to say a value that best reflects the alveolar CO₂, with the objective of better assisting the first responder during the CPR by providing him with pertinent information that facilitates monitoring of the CPR, such as the detection of a resumption of spontaneous cardiac activity (RSCA), for example.

### SUMMARY

The solution of the invention is therefore a respiratory assistance apparatus, that is to say a medical ventilator, for delivering a respiratory gas, such as oxygen, to a patient during cardiopulmonary resuscitation (CPR), comprising:
a source of respiratory gas for delivering a respiratory gas to said patient during cardiopulmonary resuscitation (CPR),
means for measuring the CO₂ content in order to perform measurements of the concentration of CO₂ produced by the patient, and to supply CO₂ content measurement signals to signal-processing and control means,
signal-processing and control means configured to process the CO₂ content measurement signals originating from the CO₂ content measurement means,
at least one graphical user interface (GUI),
   characterized in that:
the signal-processing and control means are configured:
   a) to process the CO₂ content measurement signals corresponding to measurements performed by the CO₂ content measurement means during a given time period (dt), and to extract therefrom a plurality of CO₂ content values,
   b) to select the maximum CO₂ content value (Vmax) from the plurality of CO₂ content values measured during said given time period (dt), and
   c) to transmit said maximum CO₂ content value (Vmax) to the graphical user interface,
and the graphical user interface is configured to display the maximum CO₂ content value (Vmax).

Depending on the case, the respiratory assistance apparatus of the invention may comprise one or more of the following technical features:
the GUI is configured to display at least one CO₂ content value supplied by the signal-processing and control means.
the CO₂ produced by the patient. This CO₂ can be observed during exhalation of the patient and/or re-inhaled at the following inhalation, especially if it is gas trapped between a Y-shaped piece and the CO₂ sensor, for example.
the source of respiratory gas is in fluidic communication with a gas conduit.
the source of respiratory gas is an air source, in particular a motorized micro-blower, also called a turbine or compressor.
the means for measuring the CO₂ content are arranged in such a way as to perform CO₂ concentration measurements downstream from the gas conduit.
the means for measuring the CO₂ content are connected electrically to the signal-processing and control means.
the source of respiratory gas is in fluidic communication with a gas conduit through which the respiratory gas is conveyed to the patient, i.e. as far as a respiratory interface.
the signal-processing and control means comprise at least one electronic board or similar.
the signal-processing and control means comprise at least one microprocessor, preferably a microcontroller.
the microprocessor uses at least one algorithm.
the means for measuring the CO₂ content are arranged in the main flow of gas, that is to say in a ‘mainstream’ configuration.
the means for measuring the CO₂ content comprise a capnometer.
the means for measuring the CO₂ content are arranged in such a way as to perform CO₂ concentration measurements downstream from the gas conduit, preferably at a downstream end of the gas conduit.
the gas conduit is in fluidic communication with a respiratory interface, also called a patient interface.
the respiratory interface is an endotracheal intubation tube, a face mask or a laryngeal mask, also called a supraglottic device, or any device suitable for administering gas.
the respiratory interface is preferably an endotracheal intubation tube, commonly called a “tracheal tube”.
the means for measuring the CO₂ content are arranged upstream from and in immediate proximity to the respiratory interface, that is to say near the patient's mouth.
according to a first embodiment, the means for measuring the CO₂ content are arranged on a junction piece arranged upstream from the respiratory interface, preferably between the respiratory interface and the downstream end of the gas conduit, in particular between the respiratory interface and a Y-shaped piece comprising internal passages for gas.
preferably, the means for measuring the CO₂ content are arranged on a junction piece comprising an internal passage for gas.
according to a second embodiment, the means for measuring the CO₂ content are arranged in the apparatus, that is to say in the framework of the apparatus, and are connected, via a gas sampling conduit or similar, to a gas sampling site situated upstream from and in immediate proximity to the respiratory interface.
in particular, the means for measuring the CO₂ content are connected fluidically to a gas sampling site carried by a junction piece, in particular arranged between the respiratory interface and the gas conduit, typically between the respiratory interface and a downstream end of said gas conduit.
the junction piece is attached fluidically between the intermediate attachment piece, that is to say a Y-shaped piece, and the respiratory interface, typically a tracheal tube or a mask.
the inhalation branch, the exhalation branch and the respiratory interface are in fluidic communication with each other.
it comprises a patient circuit comprising an inhalation branch through which gas can be conveyed to the patient.
the patient circuit additionally comprises an exhalation branch for discharging the gas exhaled by the patient.
the inhalation branch, the exhalation branch and the patient interface are connected fluidically and/or mechanically, directly or indirectly, to the intermediate attachment piece, in particular a Y-shaped piece.
the means for measuring the CO₂ content are arranged in such a way as to perform CO₂ concentration measurements at the inlet of the exhalation branch or at the outlet of the inhalation branch of the gas circuit.
the exhalation branch communicates fluidically with the atmosphere in order to discharge all or some of the gas exhaled by the patient, in particular the gas rich in CO₂.
the inhalation branch and/or the exhalation branch comprise flexible hoses.
preferably, all or part of the gas conduit forming all or part of the inhalation branch of the gas circuit is a flexible hose.
the signal-processing and control means are configured to control the source of respiratory gas and to deliver the respiratory gas in successive ventilatory cycles.
each ventilatory cycle comprises a phase LP (D_{LP}) during which the gas is delivered by the micro-blower at a low pressure (LP), and a phase HP (D_{HP}) during which the gas is delivered by the micro-blower at a high pressure (HP), with HP>LP.
the micro-blower is controlled to deliver gas at a low pressure (LP) of between 0 and 20 cm of water, preferably between 0 and 15 cm of water, more preferably between 0 and 10 cm of water.
the micro-blower is controlled to deliver gas at a high pressure (HP) of between 5 and 60 cm of water, preferably between 5 and 45 cm of water, more preferably between 5 and 30 cm of water (with HP>LP).
the phase LP has a duration longer than the phase HP.
the phase LP has a duration of between 2 and 10 seconds, typically of the order of 3 to 6 seconds.
the phase HP has a duration of between 0.5 and 3 seconds, typically of the order of 1 to 2 seconds.
the given time period (dt) is of several seconds.
the time period (dt) is between 2 and 10 seconds, typically of the order of 3 to 6 seconds.
the time period (dt) corresponds to the duration of the phase LP of each ventilatory cycle.
the total duration of a ventilatory cycle is between 3 and 10 seconds.
the given time period (dt) encompasses several durations of successive chest compressions and relaxations, typically between 5 and 12 chest compressions.
the means for measuring the CO₂ content are configured to perform measurements continuously.
the means for measuring the CO₂ content comprise a CO₂ sensor.
the means for measuring the CO₂ content comprise a CO₂ sensor whose measuring tap is in fluidic communication with the interior or lumen of the junction piece arranged upstream from the respiratory interface.
it comprises storage means cooperating with the signal-processing and control means in order to store the plurality of CO₂ content values measured during the given time period.
the storage means are additionally configured to store the successive maximum CO₂ content values (Vmax), that is to say the values measured during successive given time periods (dt).
the storage means comprise a flash memory or hard disk memory.
it additionally comprises means for measuring the gas flow rate, which are configured to perform at least one measurement, preferably continuously, of the flow rate of the gas exhaled and/or the flow rate of gas inhaled by the patient. The flow rate permits monitoring of the chest compressions, and also the calculation and monitoring of the volumes of gas that are delivered and exhaled (ventilator and chest compressions).
the means for measuring the flow rate of gas comprise a flow rate sensor.
the graphical user interface (GUI) comprises a digital screen, preferably a touch screen.
the graphical user interface is configured to display the maximum CO₂ content value (Vmax) in the form of a numerical value or of a graphical representation displayed on the GUI, or both.
the graphical user interface is configured to display several successive maximum CO₂ content values (Vmax) in the form of a graphical representation.
the graphical user interface is configured to display one or more successive maximum CO₂ content values (Vmax) in the form of a graphical representation such as a curve, bar graph or similar.
the screen comprises several touch controls that activate different functions and/or several display zones or windows.
the screen is of the type with colour display.
alternatively, the screen is of the type with black and white display or permits a change-over from colour display to black and white display in order to save energy.
it comprises a source of electric current, for example a battery or similar, preferably a rechargeable battery.
it comprises alarm means which are configured to trigger when the maximum CO₂ content value exceeds a threshold value.
the alarm means comprise an acoustic or visual alarm, or both.
the alarm means are programmed to trigger when the maximum value (Vmax) of CO₂ measured at a time t is such that: [VmaxCO₂]>n×[MeanCO₂], where:
   n is between 1.25 and 4, preferably between 1.5 and 3, for example of the order of 2,
   [VmaxCO₂] is the maximum CO₂ content value measured during a given duration dt, for example over a duration dt of between 2 and 10 seconds,
   [MeanCO₂] is the mean value of the maximum CO₂ content values [VmaxCO₂] determined for several successive durations dt in a given time window (FT) (FT>x.dt with x≥2), for example a period of 30 seconds to 5 minutes, or more.
it comprises a rigid framework comprising the source of respiratory gas, the signal-processing and control means, the source of electric current and the storage means.
the rigid framework is formed wholly or partly of polymer.
the graphical interface is arranged in one of the walls forming the framework of the ventilator.
the means for measuring the CO₂ content are configured to perform successive measurements of CO₂ concentration over successive time periods (dt), that is to say time periods (dt) spaced apart from one another.
the means for measuring the CO₂ content are configured to perform successive measurements of CO₂ concentration over successive time periods (dt) during successive ventilatory cycles, in particular during the LP phases of successive ventilatory cycles.

The invention also relates to a method for monitoring cardiopulmonary resuscitation (CPR) performed on a patient in cardiac arrest, in which method:
use is made of a respiratory assistance apparatus comprising a source of respiratory gas, such as a micro-blower, in order to deliver a respiratory gas to a patient during cardiopulmonary resuscitation (CPR),
measurements of the concentration of CO₂ produced by said patient are performed, for example by means of a capnometer,
the CO₂ content measurement signals are processed, for example by signal-processing and control means such as a microprocessor,
a plurality of CO₂ content values measured during a given time period (dt) are determined,
the maximum CO₂ content value (Vmax) is selected from the plurality of CO₂ content values,
the maximum CO₂ content value (Vmax) is displayed, during the given time period (dt), on a GUI.

### BRIEF DESCRIPTION OF THE DRAWINGS

The invention will now be better understood from the following detailed description given as a non-limiting example and with reference to the appended figures, in which:

FIG. 1 is a graphical representation of the variations of the CO₂ content in the respiratory gases of a patient who is being ventilated and who is not in cardiac arrest,

FIG. 2 is a diagram showing a ventilatory cycle with two pressure levels that can be used by the apparatus of FIG. 6 in order to ventilate a patient in cardiopulmonary arrest during CPR,

FIG. 3 illustrates the pressure variations observed by the machine at the end of the respiratory circuit in the case of a patient in cardiopulmonary arrest during CPR,

FIG. 4 is a diagram showing the quantity of CO₂ measured by the capnometer of the apparatus of FIG. 6 before and after a resumption of spontaneous cardiac activity,

FIG. 5 is a diagram showing the CO₂ content peaks during the ventilatory cycles implemented during CPR, and

FIG. 6 is a diagram showing an embodiment of a respiratory assistance apparatus for CPR according to the invention.

FIG. 6 is a schematic representation of an embodiment of a respiratory assistance apparatus or medical ventilator according to the invention used for delivering a respiratory gas, typically air or oxygen-enriched air, to a patient P during cardiopulmonary resuscitation (CPR), that is to say to a person who is in cardiac arrest and on whom a first responder performs cardiac massage, that is to say an alternation of chest compressions (CC) and relaxations (Re), that is to say non-compressions.

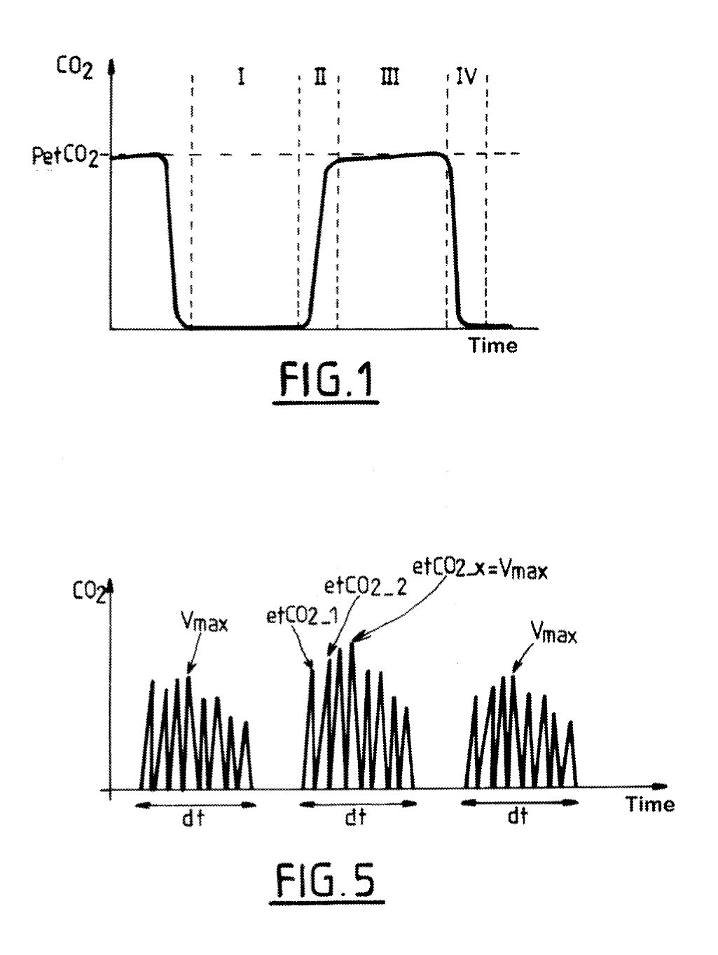

### DESCRIPTION OF PREFERRED EMBODIMENTS

This apparatus or ventilator comprises a source 1 of respiratory gas, such as a motorized micro-blower, which is in fluidic communication with a gas conduit 2 for delivering a respiratory gas to said patient P during cardiopulmonary resuscitation, typically pressurized air.

The source 1 of respiratory gas is governed, that is to say controlled, by signal-processing and control means 5, in particular an electronic board with microprocessor 6 or similar. The signal-processing and control means 5 control the source 1 of respiratory gas in such a way that it delivers the gas in accordance with one or more predefined ventilation modes.

Preferably, the signal-processing and control means 5 make it possible to control the source 1 of respiratory gas so as to deliver the gas in accordance with a “normal” ventilatory mode, corresponding to ventilation of a patient who is not in cardiac arrest, and a “CPR” ventilatory mode, corresponding to ventilation of a patient who is in cardiac arrest and on whom a first responder initiates or performs CPR.

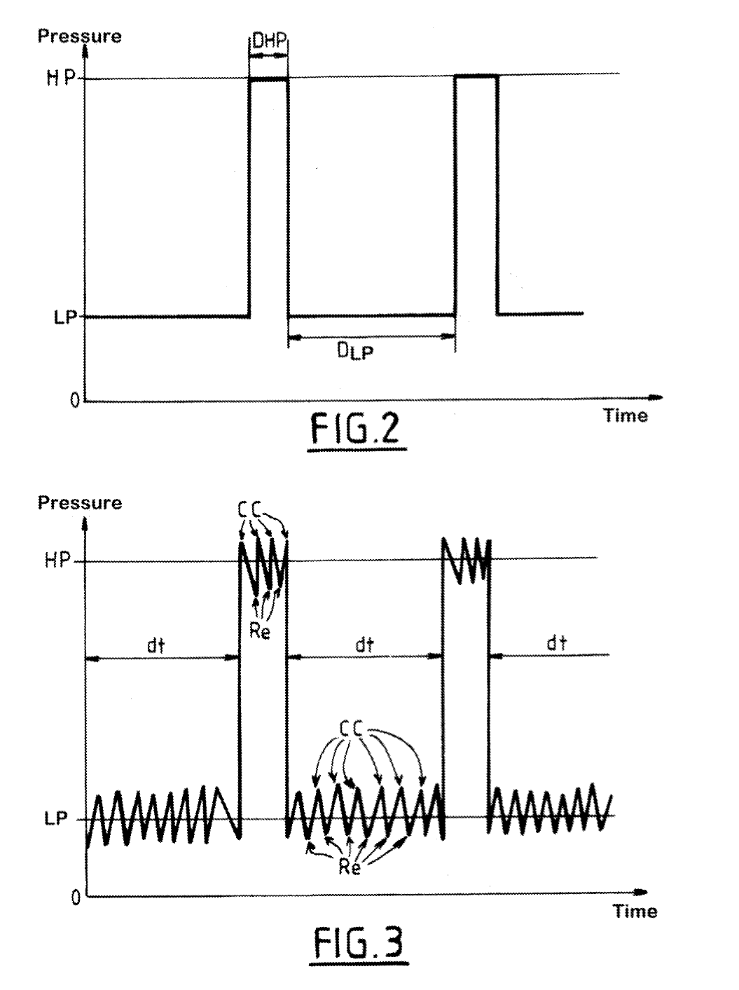

For example, in accordance with a ventilation mode intended for CPR, the source 1 of respiratory gas is controlled so as to deliver the respiratory gas, typically air, in a ventilatory cycle comprising several pressure levels or of the BiPAP type, as illustrated in FIG. 2, in particular two pressure levels comprising a low pressure level, for example a low pressure (LP) of between approximately 0 cm H₂O and 15 cm H₂O, and a high pressure level, for example a high pressure (HP) of between approximately 7 cm H₂O and 40 cm H₂O.

The gas is delivered alternately between these two pressure levels (LP, HP), as is illustrated in FIG. 2, throughout the CPR performed by the first responder, that is to say while the first responder performs the chest compressions and relaxations. The duration (D_{LP}) of delivery of gas at low pressure (LP) by the micro-blower 1 is between 2 and 10 seconds, typically of the order of 3 to 6 seconds, whereas the duration (D_{HP}) of delivery of gas at high pressure (HP) is less than 3 seconds, for example of the order of 0.5 to 1.5 seconds.

The micro-blower 1 of the ventilator generates two pressure levels, namely a high-pressure level (i.e. HP) and a low-pressure level (i.e. LP). The cardiac massage alternating between phases of chest compression (CC) and relaxation (Re) generates pressure peaks, which are superposed on the pressure cycles of the ventilator. This results, at the patient interface, in a pressure curve as illustrated in FIG. 3 where the pressure peaks at the high plateaus (i.e. at HP) and low plateaus (i.e. at LP) reflect the chest compressions (CC) with increased pressure, since the chest yields under the pressure of the CC performed by the first responder, and the relaxations (Re) with low pressure, since the chest rises again in the absence of CC.

As will be seen from FIGS. 2 and 3, in the context of the present invention the given time period (dt), during which the plurality of CO₂ content values are measured and the maximum CO₂ content value (Vmax) is extracted therefrom, corresponds to the duration (DLP) of delivery of gas at low pressure (LP), i.e. between 2 and 10 seconds, typically between 3 and 6 seconds.

The gas delivered by the micro-blower 1 is conveyed through the gas conduit 2 which forms all or part of the inhalation branch 2a of the patient circuit 2a, 2b. The respiratory gas, generally air, is delivered to the patient via a gas distribution interface 3, for example here an endotracheal intubation tube, more simply called a tracheal tube. However, other interfaces may be used, in particular a face mask or a laryngeal mask.

The gas conduit 2 is in fluidic communication with the gas distribution interface 3, such as a tracheal tube, in such a way as to supply the latter with the gas originating from the source 1 of respiratory gas, in this case a micro-blower. The gas conduit 2 will in fact be attached to the tracheal tube 3 by way of an intermediate attachment piece 8, here a Y-shaped piece. This Y-shaped intermediate attachment piece 8 comprises internal passages for gas.

The intermediate attachment piece 8, that is to say the Y-shaped piece, is likewise attached to the exhalation branch 2b of the patient circuit 2a, 2b so as to be able to collect and convey the gases rich in CO₂ that are exhaled by the patient P and to discharge them to the atmosphere (at 9).

Also provided according to the invention are means 4 for measuring the CO₂ content, called a CO₂ sensor or capnometer, which means are designed to perform measurements of the concentration of CO₂ in the gas exhaled by the patient P and to deliver CO₂ content measurement signals to the signal-processing and control means 5, where these measurement signals can be processed, in particular by one or more calculation algorithms or similar.

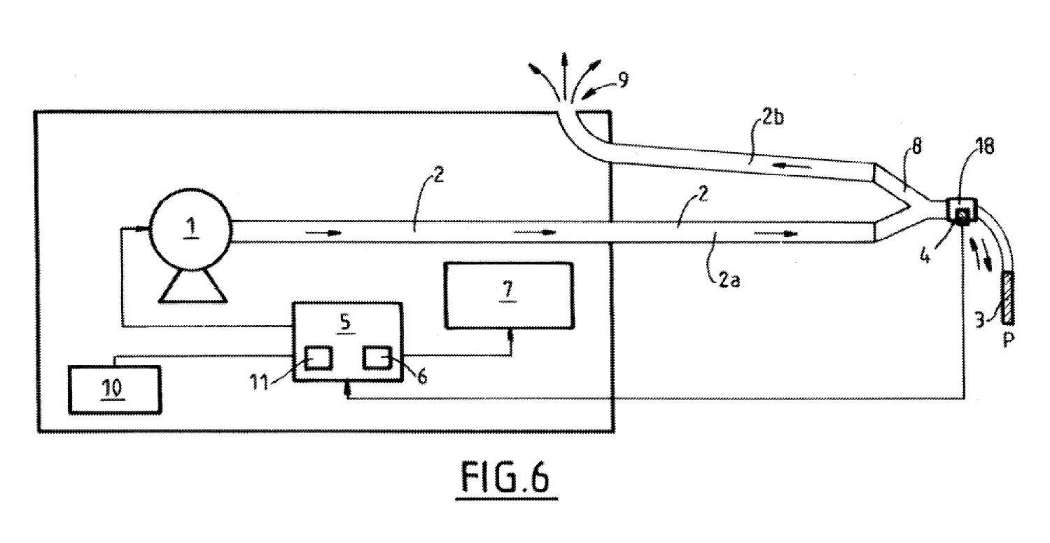

In the embodiment in FIG. 6, the CO₂ sensor is arranged near the mouth of the patient P in the mainstream configuration, that is to say upstream from and in immediate proximity to the respiratory interface 3, preferably between the intermediate attachment piece 8, i.e. the Y-shaped piece, and the respiratory interface 3, i.e. the tracheal tube, for example on a junction piece 18 (cf. FIG. 6).

According to another embodiment (not shown), the CO₂ sensor can be arranged in the “sidestream” configuration. In this case, the CO₂ sensor 4 is situated in the framework of the respiratory assistance apparatus and is connected, via a gas sampling line, such as tubing or the like, to a gas sampling site situated upstream from and in immediate proximity to the respiratory interface 3, for example on the junction piece 18. This gas sampling line communicates fluidically with the lumen of the junction piece 18 in such a way as to be able to collect a sample of the gas from there and convey it then to the CO₂ sensor situated in the framework of the apparatus.

In all cases, the junction piece 18 comprises an internal passage for gas, allowing the gas to pass through it.

Preferably, the CO₂ sensor performs continuous measurements of the concentration of CO₂ in the gas flowing through the junction piece 18, which gas is enriched in CO₂ during its passage through the lungs of the patient P, where gaseous exchanges take place.

The CO₂ content measurement signals are then transmitted by the CO₂ sensor to the signal-processing and control means 5 by an electrical connection or similar, in particular by wire or similar.

The monitoring of the CO₂ content, in particular of the etCO₂ which indirectly reflects the alveolar CO₂ content, is in fact of great importance during CPR, especially for detecting a resumption of spontaneous cardiac activity (RSCA). This is because a resumption of spontaneous cardiac activity (RSCA), hence a significant increase of the cardiac output, brings about a rapid increase in the quantity of CO₂ carried by the blood to the lungs and transferred through the alveolar-capillary membrane, this CO₂ then being found again in the gas flow exhaled by the patient.

The signal-processing and control means 5 (in particular the microprocessor 6) are configured:

a) to process the CO₂ content measurement signals corresponding to measurements performed by the CO₂ content measurement means 4, typically the capnometer or CO₂ sensor, during the given time period (dt), for example several seconds, in order to extract therefrom a plurality of CO₂ content values.

b) to select the maximum CO₂ content value (Vmax) from the plurality of CO₂ content values measured during said given time period (dt), and

c) to transmit this maximum CO₂ content value (Vmax) to a graphical user interface 7 or GUI.

A source 10 of electric current, such as a rechargeable battery or similar, directly or indirectly supplies electric current to the signal-processing and control means 5, the micro-blower 1, the GUI 7 or any other element of the apparatus, in particular a storage memory 11. The source 10 of electric current is preferably arranged in the framework of the ventilator.

Generally, the medical ventilator of the invention permits a continuous measurement of the concentration of CO₂ produced by the patient P, the measurement being performed by the capnometer 4 which is arranged on the pathway of the gas, close to the mouth of the patient P, preferably here between the Y-shaped piece 8 and the tracheal tube 3 of FIG. 6, that is to say at the junction piece 18 attached fluidically between the Y-shaped piece 8 and the tube 3.

If so desired, the ventilator additionally permits parallel performance of a continuous measurement of the exhaled and inhaled gas flow rates, with the aid of one or more flow rate sensors (not shown).

According to the invention, the GUI for its part is configured to display the maximum CO₂ content value supplied by the signal-processing and control means 5, which value is selected from several CO₂ concentration values measured for a given duration corresponding to several successive chest compressions and relaxations performed by a first responder carrying out cardiac massage (i.e. CPR) on the patient P in cardiac arrest.

The reason is that the CO₂ concentration value which best reflects the alveolar CO₂ content, and which hence gives a good indication of the state of the blood flow in the patient P during the CPR, is the highest CO₂ value, also called the maximum value (Vmax) or peak value, as illustrated in FIG. 5 which shows the development of the CO₂ content in the gas and illustrates several etCO₂ measurements for several successive durations (dt), for example durations of 3 to 6 seconds, while CPR is being performed. It will be seen here that the CO₂ content of the gas is not constant during a given time interval dt and that there is therefore necessarily a maximum CO₂ content value (Vmax) over each interval dt, that is to say the peak value.

Hence, in the context of the present invention, the ventilator thus stores (at 11) all the peak values of CO₂ during each time period dt, typically between 3 and 7 seconds, and determines the maximum CO₂ content value (Vmax) from the plurality of peaks (EtCO2_{—1}, EtCO2_{—2}, EtCO2_{—3}, . . . , EtCO2_{—x}) measured over a given time period, as is illustrated in FIG. 5.

During CPR, the CO₂ content in the gas produced by the patient, and passing the measurement tap of the capnometer 4, varies depending on the presence or absence of chest compressions (CC).

Thus, after insufflation of air by the micro-blower 1 of the ventilator and as long as chest compression has not commenced, no CO₂ is detected in the gas flows passing through the conduit 2 as far as the respiratory interface 3, which then distributes this air to the lungs of the patient P.

After several chest compressions (CC) performed by a first responder, CO2 is detected at the Y-shaped piece 8 by the capnometer 4 since the alternations of chest compressions (CT) and relaxations (Re) generate movements of air entering and leaving the lungs of the patient P by “imitating” the exhalation phases of the patient P. Exhaled air rich in CO₂ is then found again at the Y-shaped piece 8 and the capnometer 4 (cf FIG. 6), and measurements of the CO₂ concentrations can be performed by the capnometer 4. The corresponding measurement signals are sent to the signal-processing and control means 5 where they are processed in the way explained above, so as to determine the maximum CO₂ content value (Vmax) over each time interval dt.

The maximum CO₂ value (Vmax) is the one that best represents the alveolar CO₂. In fact, the CO₂ present at the Y-shaped piece 8 and the capnometer 4 is “washed out” little by little on account of the successive and repeated chest compressions and tends to decrease after reaching this maximum value, since the chest compressions thus cause the discharge to the atmosphere (at 9) of the gases rich in CO₂, via the exhalation branch 2b of the patient circuit. The successive chest compressions (CC) thus generate different levels of CO₂, the most representative one being the peak value or maximum value (Vmax), as is illustrated in FIG. 5.

In the context of the present invention, the ventilator thus stores (at 11) all the maximum CO₂ content values (Vmax) between two ventilatory cycles, that is to say during the successive durations dt, determines the maximum CO₂ content value (Vmax) from the plurality of maximum values measured, and displays this maximum value (Vmax) on the screen of the GUI 7.

This maximum value (Vmax), during a given time interval dt, can be displayed as a single numerical value. It is also possible to display several maximum values (Vmax) measured successively over several successive time intervals (dt). Furthermore, if it is deemed useful or desirable, it is also possible to display the value in the form of a graphical representation showing several maximum values (Vmax) measured successively over several successive time intervals (dt) over the course of time, for example over the last 2 to 5 minutes, for example a graphical representation such as a curve, bar graph or similar.

The data calculated from these CO₂ measurements allow the first responder to better “control” the CPR, by virtue of an indicator which reflects the state of the circulation and metabolism of the patient since, at a constant ventilation level, the more effective the CPR, the greater the quantity of CO₂ produced and transferred through the alveolar-capillary membrane, hence the greater the quantity of CO₂ that can be detected at the capnometer 4.

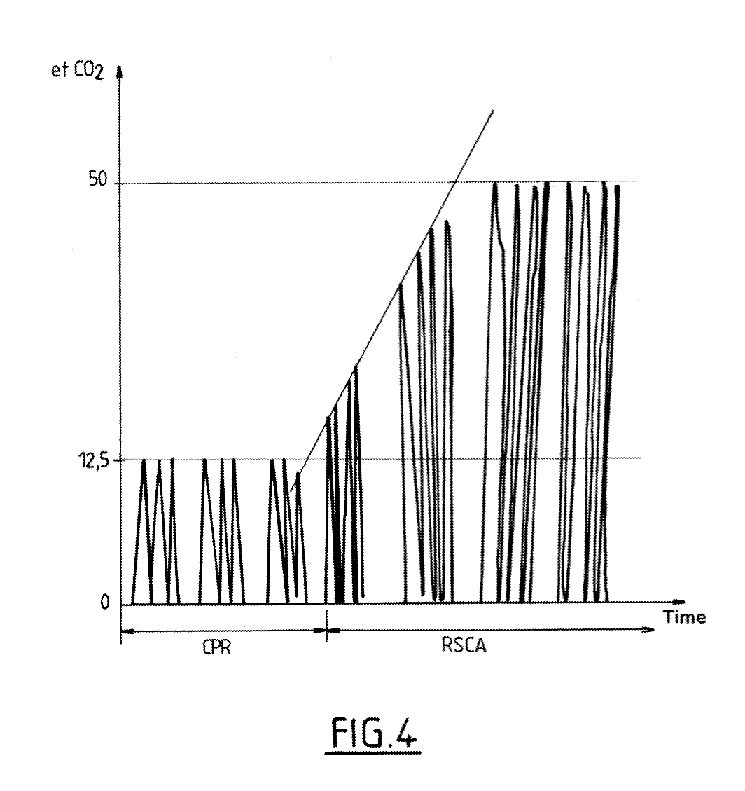

Hence, in the case of a resumption of spontaneous cardiac activity (RSCA), the circulation recovers abruptly and therefore the quantity of alveolar CO₂ increases in parallel, which induces a substantial increase in the quantity of CO₂ detected by the capnometer 4 by a factor often greater than 2, as is illustrated in FIG. 4. It will in fact be seen from FIG. 4 that the etCO₂ is always below 25 KPa during the CPR, whereas the etCO₂ increases suddenly to reach over 50 KPa in the event of resumption of spontaneous cardiac activity (RSCA). This can be immediately detected by the first responder, who can then carry out an analysis of the heart rhythm in order to stop cardiac massage in the case of effective RSCA.

To put it another way, in the context of the invention, the fact that the GUI 7 displays the maximum etCO₂ value, during a given time period (dt), allows the first responder to better detect the occurrence of an RSCA since this maximum CO₂ value (Vmax) closely reflects the alveolar CO₂.

It has in fact been found, in tests carried out in the context of the present invention, that continuously displaying all the CO₂ measurements would not be effective, since the cardiac massage itself, even when carried out uniformly (pressure force, frequency, etc.), inevitably causes considerable variations in CO₂ content at the capnometer from one chest compression to another. This is explained by the dynamic behaviour or opening/closing of the small airways and by the effect of lavage of the dead space during the successive chest compressions between two machine cycles. Therefore, displaying all the CO₂ measurements could cause the first responder to make an error or could “drown” him under too much information, and he could then sometimes believe there was a resumption of spontaneous cardiac activity even when it was only an artefact, or, conversely, the first responder could fail to notice a resumption of spontaneous cardiac activity (RSCA) in the patient and could continue the massage when the patient is in the RSCA phase. In all cases, the use of a single instantaneous value for prognostic reasons or for choice of therapeutic strategy is made risky by the oscillating nature of the instantaneous etCO2 value, i.e. at each chest compression (CC).

In the context of the invention, it has been shown in practical tests that these problems could be completely overcome by displaying only the highest CO₂ content value (Vmax) during a given time period (dt), typically of a few seconds.

In addition, it has been found that the CO₂ content measured at each chest compression can vary enormously from one chest compression to another. This is due not only to the anatomical and instrumental dead space but also to the degree of opening of the patient's airways. Taking these factors into account, the maximum CO₂ content value (Vmax) appears therefore to be a better reflection of the alveolar CO₂ and is thus a good indicator of RSCA (if it increases abruptly) or of a new cardiac arrest (if its decreases abruptly), which informs the first responder immediately and in a more relevant way.

Thus, when the first responder notes a strong increase in the displayed CO₂ value, he can conclude from this that the patient is in the RSCA phase, as is illustrated in FIG. 4, and can then decide to stop the cardiac massage in order to carry out an analysis of the heart rhythm for example.

Advantageously, the ventilator of the invention can also include alarm means designed and programmed to warn the first responder or the like when the measured maximum CO₂ value exceeds or, conversely, drops below a given value that is predefined or calculated continuously.

In particular, an acoustic and/or visual alarm is provided which triggers when the maximum CO₂ content measured, at a time t, is greater than a threshold value, for example: [VmaxCO₂]>1.5×[MeanCO₂] where:
[VmaxCO₂] is the maximum CO₂ content value measured during a given duration dt, for example over a duration dt of between 2 and 10 seconds,
.[MeanCO₂] is the mean value of the maximum CO₂ content values [VmaxCO₂] determined for several successive durations dt in a given time window (FT) (FT>x.dt with x≥2), for example a period of 30 seconds to 5 minutes, or more.

Similarly, the alarm can trigger in the event of the CO₂ concentration dropping abruptly below a given minimum value, which could be the sign of a new cardiac arrest of the patient, of hyperventilation, or of obstruction of the gas circuit between the patient and the machine, for example a flexible conduit that is bent or crushed and no longer allows the gas to pass through.

Generally, the invention relates to a medical ventilator suitable for use during cardiopulmonary resuscitation (CPR), comprising a source 1 of respiratory gas, such as a micro-blower, means 4 for measuring the CO₂ content, such as a capnometer, signal-processing and control means 5 receiving and processing the CO₂ content measurement signals originating from the CO₂ content measurement means 4, and a GUI 7 configured to display at least one maximum CO₂ content value (Vmax) measured during a given time period (dt), said maximum CO₂ content value (Vmax) being selected from a plurality of CO₂ content values measured during said given time period (dt).

The respiratory assistance apparatus or medical ventilator according to the present invention is particularly suitable for use during cardiopulmonary resuscitation (CPR) on a person (i.e. a patient) in cardiopulmonary arrest, in the context of which a respiratory gas such as pressurized air is supplied, in accordance with a ventilatory cycle with several pressure levels, to said person undergoing the cardiac massage with alternating chest compressions and relaxations. To facilitate its transport by the first aid responders, for example by a physician, a nurse, a fire fighter or similar, the ventilator of the invention is preferably arranged in a bag for carrying it.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A respiratory assistance apparatus for delivering a respiratory gas to a patient during cardiopulmonary resuscitation (CPR), comprising:
a source (1) of respiratory gas for delivering a respiratory gas to said patient during cardiopulmonary resuscitation (CPR),
a CO₂ content measurement device (4) adapted to measure the CO₂ content in order to perform measurements of the concentration of CO₂ produced by said patient, and to supply CO₂ content measurement signals to a signal-processing and control system (5),
a signal-processing and control system (5) configured to process the CO₂ content measurement signals originating from the CO₂ content measurement device (4),
at least one graphical user interface (7),
wherein:
the signal-processing and control system (5) is configured:
a) to process the CO₂ content measurement signals corresponding to measurements performed by the CO₂ content measurement device (4) during a given time period (dt), and to extract therefrom a plurality of CO₂ content values,
b) to select the maximum CO₂ content value (Vmax) from the plurality of CO₂ content values measured during said given time period (dt), and
c) to transmit said maximum CO₂ content value (Vmax) to the graphical user interface (7),
and wherein the graphical user interface (7) is configured to display the maximum CO₂ content value (Vmax).

2. The apparatus according to claim 1, wherein the signal-processing and control system (5) comprises at least one microprocessor.

3. The apparatus according to claim 1, wherein the CO₂ content measurement device (4) comprises a capnometer.

4. The apparatus according to claim 1, wherein a gas conduit (2) is in fluidic communication with a respiratory interface (3).

5. The apparatus according claim 1, wherein the CO₂ content measurement device (4) is arranged:
either upstream from and in immediate proximity (18) to a respiratory interface (3),
or in the apparatus, being connected to a gas sampling site (18) situated upstream from and in immediate proximity to the respiratory interface (3).

6. The apparatus according to claim 1, wherein the given time period (dt) is between 2 and 10 seconds.

7. The apparatus according to claim 1, the CO₂ content measurement device (4) is configured to perform measurements continuously.

8. The apparatus of claim 1, further comprising a storage device (8) cooperating with the signal-processing and control system (5) in order to store the plurality of CO₂ content values measured during the given time period.

9. The apparatus according to claim 1, further comprising an alarm configured to trigger when the maximum CO₂ content value exceeds a threshold value.

10. The apparatus according to claim 1, wherein the graphical user interface (GUI) comprises a digital screen.

11. The apparatus according to claim 1, wherein the signal-processing and control system (5) is configured to control the source (1) of respiratory gas and to deliver the respiratory gas in successive ventilatory cycles.

12. The apparatus according to claim 11, wherein the source (1) of respiratory gas comprises a motorized micro-blower.

13. The apparatus according to claim 1, wherein the graphical user interface (7) is configured to display the maximum CO₂ content value (Vmax) in the form of a numerical value and/or a graphical representation.

14. The apparatus according to claim 1, wherein the CO₂ content measurement device (4) is configured to perform successive measurements of the CO₂ concentration over successive time periods (dt).
